(19) United States
(12) Patent Application Publication
(10) Pub. No.: US 2005/0003007 A1
(43) Pub. Date: Jan. 6, 2005

(54) Method of sterilization of polymeric microparticles

(76) Inventors: Michele Boix, Cantaron (FR); Christian Sarrazin, Pegomas (FR); Patrick M. Hughes, Aliso Viejo, CA (US); Marina Do, Menton (FR); Isabelle Maroteaux, Antibes (FR)

Correspondence Address: BRENT A. JOHNSON;ALLERGAN, INC., T2-7H, 2525 Dupont Drive, Irvine, CA, 92612, US

(21) Appl. No.: 10/614,408

(22) Filed: Jul. 2, 2003

(51) Int. Cl. A61K 9/14

(52) U.S. Cl. 424/486

(57) ABSTRACT

This invention relates to the sterilization of polymeric material by irradiation for use in a body of a mammal. We have surprisingly discovered that the sterilization of polymeric materials for use in a mammal by irradiation is improved by reducing the temperature at which the irradiation is carried out. One aspect of this invention relates to a sterilized polymeric material for use in a body of a mammal wherein said polymeric material is sterilized by irradiation at a reduced temperature Another aspect of this invention relates to methods of sustained delivery of a therapeutically active agent to a mammal by using a polymeric material that has been sterilized at a reduced temperature by irradiation.

### FIELD OF THE INVENTION

This invention relates to polymeric materials used for biological purposes. More particularly, this invention relates to method of sterilization of polymeric materials used for therapeutic purposes in a mammal.

### BACKGROUND OF THE INVENTION

### Description of Related Art

Polymeric materials are often used for therapeutic purposes in mammals in a variety of forms including prosthetic implants and devices, sutures, and drug delivery systems. Drug delivery systems incorporating polymeric materials are of particular interest in the art because they are useful in providing controlled and/or sustained release of a therapeutically active agent for the treatment of a disease or condition afflicting a person or animal. Typically, the therapeutically active agent is incorporated into the polymeric material so that it is slowly released by mechanisms such as degradation or dissolution of the polymer, erosion, diffusion, ion-exchange, or a combination thereof.

Polymeric drug delivery systems are well known in the art. They come in a variety of forms including microparticles (which comprise microspheres and microcapsules) and implants. Broadly speaking, microparticles are polymeric particles having physical dimensions in the micrometer range or smaller. Microspheres are a special class of microparticles which are monolithic and have a spherical, or nearly spherical shape. Microcapsules have an inner core comprising the therapeutically active agent and a polymeric coating on the exterior. Polymeric particles can also have dimensions significantly smaller than the micrometer range, and these are sometimes called nanoparticles, nanospheres, or nanocapsules. However, for the sake of simplicity, the term microparticles will be used herein to refer to any polymeric particle of a diameter of about 100 micrometers or smaller. An implants is a polymeric drug delivery system having a macroscopic size, which could be in any shape or physical form. For example, an implant could comprise several microspheres or microcapsules held together by any number of means, an implant could be monolithic, or an implant could have two or more distinct parts of different compositions.

In order to use a polymeric material, including a drug delivery system, in the body of a person or mammal, the polymeric material must be sterile. Sterilization is carried out by chemical treatment (such as by ethylene oxide gas), heat treatment, filtration, irradiation, or other methods. Each of these methods has limitations since most methods devised to kill pathogens can also potentially affect the chemical or physical properties of a polymer or a therapeutically active agent. Consequently, the method of sterilization is chosen considering factors such as the polymeric materials used, the identity of any active agents used, and the particular use of the polymeric material in a human or animal body. As a result, the improvement of any sterilization method for polymeric materials is a significant contribution to the art.

During irradiation, microspheres, microparticles, and microcapsules tend to agglomerate and aggregate, reducing their therapeutic usefulness. In the case of polymeric materials comprising particles, the diffusion and degradation properties of the particles are dependent on surface area to volume relationships, which are affected by aggregation. As such, surface area changes encountered with aggregation will cause significant variability in drug release and particle degradation profile. In addition, the chemical and/or physical changes effected by gamma irradiation may affect the diffusion and degradation properties of polymeric materials in other ways. Gamma irradiation also tends to have an adverse effect on the drug deliver properties of implants.

Of particular interest is the improvement of sterilization of poly lactide-co-glycolide (PLGA) and poly lactic acid (PLA) microparticles for drug delivery, which tend to aggregate or agglomerate during gamma irradiation.

### SUMMARY OF THE INVENTION

We have surprisingly discovered that the sterilization of polymeric materials for use in a body of a mammal by irradiation is improved by reducing the temperature at which the irradiation is carried out. One aspect of this invention relates to a sterilized polymeric material for use in a mammal wherein said polymeric material is sterilized by irradiation at a temperature below 25° C.

Another aspect of this invention relates to a method of sustained delivery of a therapeutically active agent to a mammal comprising administering a sterilized polymeric material comprising said therapeutically active agent to said mammal, wherein the polymeric material is sterilized by irradiation at a temperature below 25° C.

Another aspect of this invention relates to methods of sterilizing a polymeric material for use in a mammal comprising irradiating said polymeric material at a temperature below 25° C.

Another aspect of this invention relates to a composition comprising sterilized polymeric microparticles and a therapeutically active agent for use in a body of a mammal wherein said polymeric material is sterilized by irradiation with external cooling of said polymeric material during sterilization.

Another embodiment of this invention relates to a method of sterilizing a polymeric material for use in a body of a mammal comprising irradiating said polymeric material with external cooling of the polymeric material.

### BRIEF DESCRIPTION OF THE DRAWING FIGURES

FIG. 1 is a microscopic image of three batches of polylactide-co-glycolide (PLGA) microspheres sterilized at room temperature and at <5° C.

FIG. 2 is a histogram of the particle diameter of batch 1 of the microspheres before sterilization, sterilized at room temperature, sterilized at <5° C. (Cold Pack), and an overlay of the batch sterilized at <5° C. and the batch before sterilization.

FIG. 3 is histogram of the particle diameter of batch 2 of the microspheres before sterilization, sterilized at room temperature, sterilized at <5° C. (Cold Pack), and an overlay of the batch sterilized at <5° C. and the batch before sterilization.

FIG. 4 is histogram of the particle diameter of batch 3 of the microspheres before sterilization, sterilized at room temperature, sterilized at <5° C. (Cold Pack), and an overlay of the batch sterilized at <5° C. and the batch before sterilization.

### DETAILED DESCRIPTION OF THE INVENTION

The term polymeric material has the meaning generally understood in the art, and could be in any form useful for therapeutic purposes in a mammal, including, but not limited to prosthetic implants and devices, sutures, and drug delivery systems. Preferably, the polymeric material is used for drug delivery, and thus comprises a therapeutically active agent. More preferably, polymeric material is suitable for sustained delivery of said therapeutically active agent.

The preferred forms of the polymeric material comprise polymeric microspheres, microparticles, microcapsules, or implants. Even more preferred are polymeric microspheres, microparticles, or microcapsules. Most preferably, polymeric microparticles are used in this invention. The term microparticle refers to any polymeric particle having a diameter or equivalent dimension of about 100 micrometers or smaller.

Chemically, the polymeric material comprises any polymeric material useful in a body of a mammal, whether derived from a natural source or synthetic. While not intending to be limiting, some examples of useful polymeric materials for the purposes of this invention include carbohydrate based polymers such as methylcellulose, carboxymethylcellulose, hydroxymethylcellulose hydroxypropylcellulose, hydroxyethylcellulose, ethyl cellulose and chitosan, hydroxy acid polyesters such as polylactide-co-glycolide (PLGA), polylactic acid (PLA), polyglycolide, polyhydroxybutyric acid, poly γ-caprolactone, poly δ-valerolactone, and polyorthoesters. Preferably, the polymer of this invention comprises polylactide-co-glycolide (PLGA) or polylactic acid (PLA). Most preferably, the polymer of this invention comprises PLOA.

The term external cooling refers to the use of cooling source on the polymeric material such that the temperature of the polymeric material is lower at the end of the sterilization process than it would be without the external cooling. External cooling of samples during irradiation is widely practiced in the physical, chemical, and biological arts. For example, x-ray crystallography, nuclear magnetic resonance, fluorescence, infrared, microwave, and other such spectroscopic techniques where the sample is irradiated are routinely carried out with external cooling at temperatures ranging from around room temperature to as low as near 0 K. Furthermore, experiments are routinely carried out by practitioners of the chemical and physical arts where samples are irradiated at temperatures ranging from room temperature down to near 0 K. While not intending to limit the scope of the invention in any way, the cooling source could be a bath of a liquid which is cooled by means of a refrigeration method, a cryogenic liquid or solid, or where the liquid is cooled before use. While not intending to limit the scope of the invention in any way, examples of useful cooling baths include ice water, which can cool to temperatures around 0° C.; a dry ice-organic solvent bath, which can cool to temperatures down to about −77° C.; liquid nitrogen, which can cool to temperatures around 77 K; or liquid helium, which can cool to temperatures of 20 K or lower. Alternatively, the cooling source could cool the entire system comprising the radiation source, the polymeric material, and any auxiliary equipment. In such a case, the cooling source could be a cooled room, a freezer or refrigerator. The cooling source could also be cold air from outdoors on a cold day, which could be pumped in, or alternatively, the sterilization could be done outdoors.

In a preferred embodiment of this invention, the temperature of said polymeric material at the end of the sterilization process is about 10° C. to about 50° C. lower than said temperature would be in the absence of external cooling. More preferably, the temperature of said polymeric material at the end of the sterilization process is about 20° C. to about 50° C. lower than said temperature would be in the absence of external cooling.

In certain embodiments, the temperature of said polymeric material at the end of the sterilization process is about 50° C. or more lower than said temperature would be in the absence of external cooling.

In other embodiments, sterilization by irradiation is carried out at a temperature below 25° C. Preferably the sterilization by irradiation is carried out at a temperature below about 15° C., more preferably, below about 10° C. In another aspect of this invention the sterilization is carried out at a temperature from −25° C. to 5° C.

The term irradiation refers to the process of exposing the sample to a form of radiation. The type and dose of the radiation used in the irradiation process can be determined by one of ordinary skill in the art by considering the type of polymeric material, the type of any therapeutically active agent that may be present, and the use for which the polymeric material is intended. While not intending to limit the scope of invention, in many cases the dose of the radiation would be similar to that used when sterilizing the sample without external cooling. If the cooling apparatus is comprised of a material that would scatter, reflect, absorb, or otherwise decrease the dose of the radiation received by the sample, the dose should be increased accordingly. While not intending to limit the scope of the invention, some examples of radiation useful in this invention include gamma radiation, alpha radiation, beta radiation, microwave radiation, and ultraviolet radiation. In the preferred embodiment of this invention the polymeric material is sterilized by gamma irradiation. In a more preferred embodiment of this invention, the sterilization is by gamma irradiation at a dose of about 1.5 to about 4.0 mRad.

In certain embodiments of this invention, a therapeutically active agent is used. A therapeutically active agent is any chemical compound which is beneficial in preventing or treating any disease or adverse condition affecting a person or mammal. While not intending to limit the invention any way, examples of therapeutically active agents that might be used in the drug delivery system of this invention are ophthalmic agents such as retinoids, prostaglandins, tyrosine kinase inhibitors, adrenoreceptor agonists or antagonists, dopaminergic agonists, cholinergic agonists, carbonic anhydrase inhibitors, guanylate cyclase activators, cannabinoids, endothelin, adenosine agonists, and neuroprotectants; analgesics/antipyretics such as aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, and meprobamate; antibiotics such as neomycin, streptomycin, chloramphenicol, cephalosporin, ampicillin, penicillin, and tetracycline; antidepressants such as nefopam, oxypertine, doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, desipramine hydrochloride, trimipramine maleate, and protriptyline hydrochloride; antidiabetics such as biguanides, hormones, and sulfonylurea derivatives; antihypertensive agents such as propanolol, propafenone, oxyprenolol, Nifedipine, reserpine, trimethaphan camsylate, phenoxybenzamine hydrochloride, pargyline hydrochloride, deserpidine, diazoxide, guanethidine monosulfate, minoxidil, rescinnamine, sodium nitroprusside, rauwolfia serpentina, alseroxylon, phentolamine mesylate, and reserpine; anti-inflammatories such as indomethacin, naproxen, ibuprofen, ramifenazone, piroxicam, cortisone, dexamethasone, fluazacort, hydrocortisone, prednisolone, and prednisone; antineoplastics such as adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, and piposulfan; antianxiety agents such as lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, and dantrolene; immunosuppressive agents such as cyclosporine, azathioprine, mizoribine, and tacrolimus; antimigraine agents such as ergotamine tartrate, propanolol hydrochloride, isometheptene mucate, and dichloralphenazone; antianginal agents such as beta-adrenergic blockers, nifedipine, diltiazem hydrochloride nitrates, nitroglycerin, isosorbide dinitrate, pentaerythritol tetranitrate, erythrityl and tetranitrate; antipsychotic agents such as haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, and prochlorperazine; antimanic agents such as lithium carbonate; antiarrhythmics such as bretylium tosylate, esmolol hydrochloride, verapamil hydrochloride, amiodarone, encainide hydrochloride, digoxin, digitoxin, mexiletine hydrochloride, disopyramide phosphate, procainamide hydrochloride, quinidine sulfate, quinidine gluconate, quinidine polygalacturonate, flecainide acetate, tocainide hydrochloride, and lidocaine hydrochloride; antiarthritic agents such as phenylbutazone, sulindac, penicillamine, salsalate, piroxicam, azathioprine, indomethacin, meclofenamate sodium, gold sodium thiomalate, ketoprofen, auranofin, aurothioglucose, and tolmetin sodium; antigout agents such as colchicine and allopurinol; anticoagulants such as heparin, heparin sodium, and warfarin sodium; thrombolytic agents such as urokinase, streptokinase, and altoplase; antifibrinolytic agents such as aminocaproic acid; hemorheologic agents such as pentoxifylline; antiplatelet agents such as aspirin, empirin, and ascriptin; anticonvulsants such as valproic acid, divalproate sodium, phenytoin, phenytoin sodium, clonazepam, primidone, phenobarbitol, phenobarbitol sodium, carbamazepine, amobarbital sodium, methsuximide, metharbital, mephobarbital, mephenytoin, phensuximide, paramethadione, ethotoin, phenacemide, secobarbitol sodium, clorazepate dipotassium, and trimethadione; antiparkinson agents such as ethosuximide; antihistamines/antipruritics such as loradatine, hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, and trimprazine tartrate; agents useful for calcium regulation such as calcitonin and parathyroid hormone; antibacterial agents such as arnikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate; antiviral agents such as interferon gamma, zidovudine, amantadine hydrochloride, ribavirin, and acyclovir; antimicrobials such as cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G potassium, penicillin G procaine, methicillin sodium, nafcillin sodium, erythromycin ethylsuccinate, erythromycin, erythromycin estolate, erythromycin lactobionate, erythromycin siearate, erythromycin ethylsuccinate, tetracycline hydrochloride, doxycycline hyclate, and minocycline hydrochloride; anti-infectives such as GM-CSF; bronchodialators such as epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate), anticholinergic agents, aminophylline, dyphylline, metaproterenol sulfate, aminophylline, mast cell stabilizers, flurisolidebeclomethasone dipropionate, beclomethasone dipropionate monohydrate, salbutamol, beclomethasone dipropionate, ipratropium bromide, budesonide, ketotifen, salmeterol, xinafoate, terbutaline sulfate, triamcinolone, theophylline, nedocromil sodium, metaproterenol sulfate, albuterol, and flunisolide; hormones such as danazol, testosterone cypionate, fluoxymesterone, ethyltostosterone, testosterone enanihate, methyltestosterone, fluoxymesterone, testosterone cypionate, estradiol, estropipate, conjugated estrogens, methoxyprogesterone acetate, norethindrone acetate, triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate, and thyroid hormones; hypoglycemic agents such as human insulin, purified beef insulin, purified pork insulin, glyburide, chlorpropamide, glipizide, tolbutamide, and tolazamide; hypolipidemic agents such as clofibrate, dextrothyroxine sodium, probucol, lovastatin, and niacin; agents useful for erythropoiesis stimulation such as erythropoietin; antiulcer/antireflux agents such as famotidine, cimetidine, and ranitidine hydrochloride; proton pump inhibitors such as omeprazole, pantoprazole, lansoprazole, and rabeprazole; antinauseants/antiemetics such as meclizine hydrochloride, nabilone, prochlorperazine, dimenhydrinate, promethazine hydrochloride, thiethylperazine, scopolamine; vitamins and other drugs such as mitotane, visadine, halonitrosoureas, anthrocyclines, ellipticine. Additionally, any of the above compounds or other pharmacologically active entity grafted onto dendrimers, polymers or shadows are other examples of therapeutically active agents. The therapeutically active agent can also be covalently bound to the polymer comprising this invention. In a preferred embodiment of this invention, the therapeutically active agent comprises a retinoid, prostaglandin, tyrosine kinase inhibitor, glucocorticoid, androgenic steroid, estrogenic steroid or non-estrogenic steroid, intracellular adhesion molecule inhibitor or an alpha-2-adrenergic agonist. In a more preferred embodiment of this invention, the therapeutically active agent comprises a retinoid. In the case that a therapeutically active agent is used, tazarotene is the most preferred therapeutically active agent.

In certain embodiments of this invention, the polymeric material is used to accomplish the sustained delivery of the therapeutically active agent. The term sustained delivery refers to the delivery of the therapeutically active agent by a system designed to increase its therapeutic half life relative to an identical therapeutically active agent without such a delivery system.

A person skilled in the art will recognize that there are many ways in which the preferences or embodiments described above can be combined to form unique embodiments. Any combination of the preferences or embodiments mentioned herein which would be obvious to those of ordinary skill in the art are considered to be separate embodiments which fall within the scope of this invention.

The best mode of making and using the present invention are described in the following examples. These examples are given only to provide direction and guidance in how to make and use the invention, and are not intended to limit the scope of the invention in any way.

### EXAMPLE 1

Microsphere Preparation

Unless otherwise indicated, all procedures in this and other examples were carried out at room temperature (25° C.).

Batch 1

Formula: Five-Gram Batch Size

| Component | Use | Quantity |
|---|---|---|
| Phase I | | |
| Polyvinyl Alcohol (PVA) | Stabilizer | 47.5 grams |
| Purified Water | Solvent | 1600 mL |
| Phase II | | |
| Tazarotene | Active | 0.5 grams (10%) |
| Poly lactide-co-glycolide | Polymer/Vehicle | 4.50 grams |
| 75:25 intrinsic viscosity | | |
| (i.v.) 0.43 | | |
| Methylene Chloride | Solvent | 300 mL |

In a five-liter beaker a solution of 3.0% PVA is manufactured using a high shear impeller and a stirring rate of 400 to 500 rpm at 80° C. Once the PVA is in solution, the stirring rate is reduced to 200 RPM to minimize foaming. PLGA is then dissolved in the methylene chloride. Once the PLGA is in solution, tazarotene is added and brought into solution.

Microspheres are then manufactured using a solvent evaporation technique. The PVA solution is vigorously stirred while slowly adding the tazarotene/PLGA solution. The emulsion is then allowed to stir over 48 hours to remove the methylene chloride. The microspheres are then rinsed and finally freeze dried. The microspheres are frozen at −50° C., then they are freeze dried for at least 12 hours at a 4 mbar minimum pressure (400 Pa).

Batch 2

Batch 2 was prepared as described for Batch 1 except that no tazarotene was added.

Batch 3 (Unloaded)

Batch 3 was prepared as described for Batch 1 except that a 0.65 intrinsic viscosity (i.v.) 75:25 PLGA was used.

TABLE 1

| Group/ | | | |
|---|---|---|---|
| Batch | Batch 1 | Batch 2 (unloaded) | Batch 3 |
| Group 1 | not sterilized | not sterilized | not sterilized |
| (Control) | | | |
| Group 2 | sterilized at < 5° C. | sterilized at < 5° C. | sterilized |
| | | | at < 5° C. |
| Group 3 | sterilized at 25° C. | sterilized at 25° C. | sterilized |
| | | | at 25° C. |

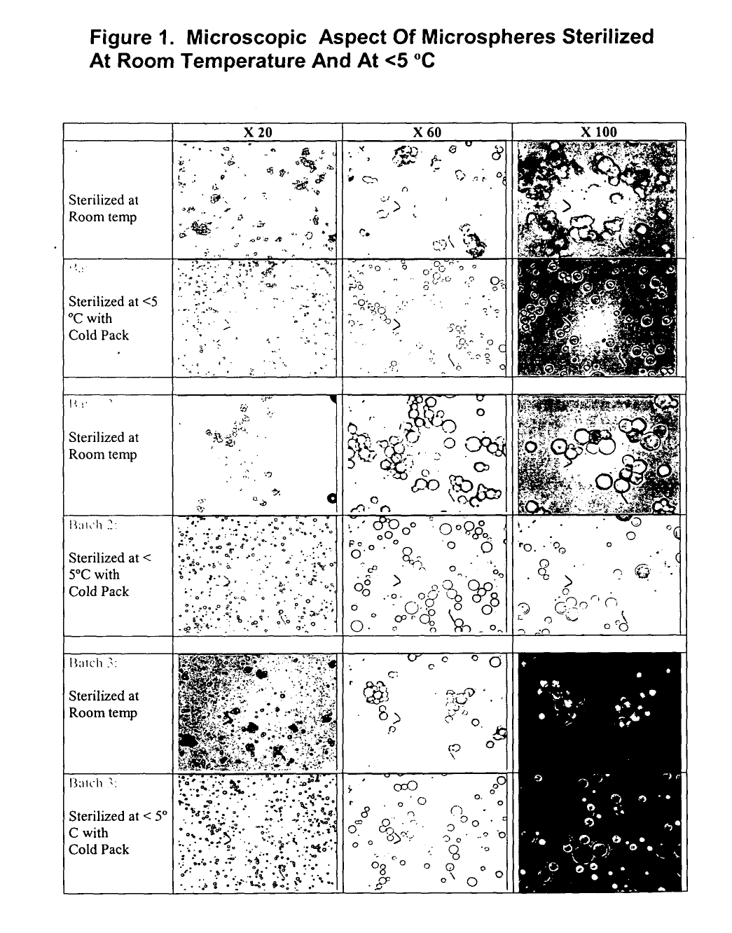

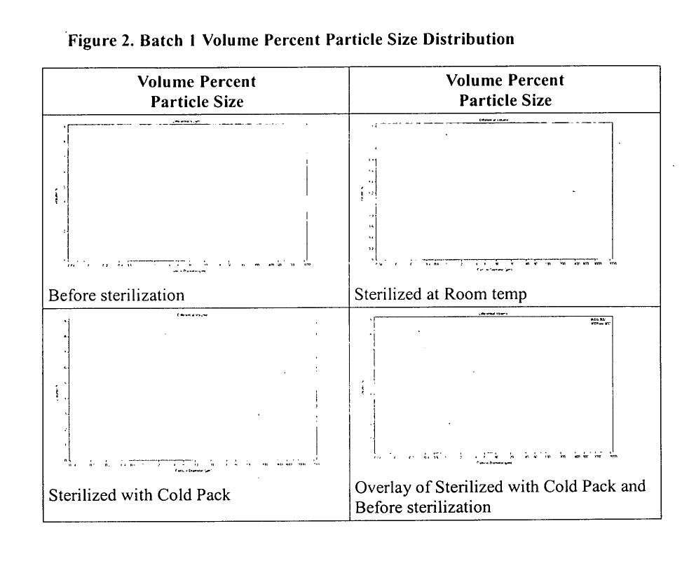

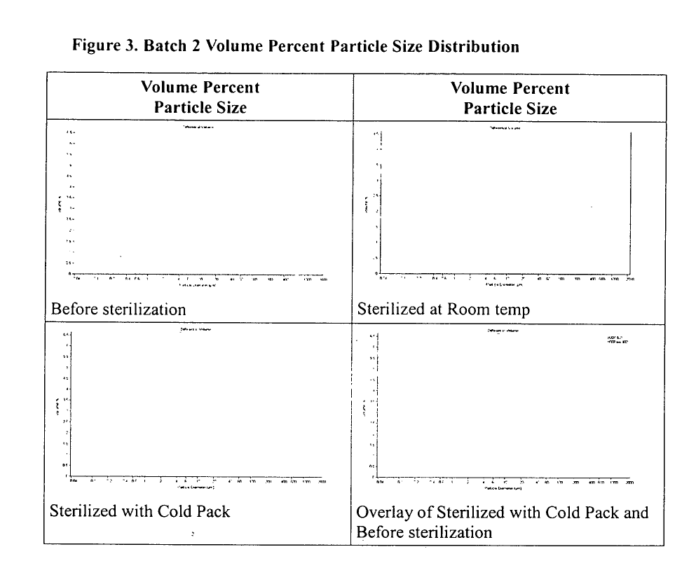

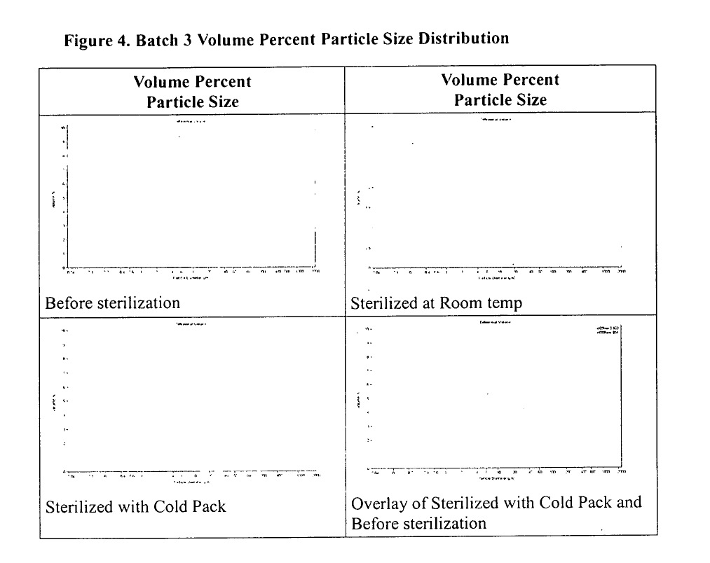

The freeze-dried microspheres were then sterilized. Each of the two batches were divided into three groups, as depicted in Table 1. The first group, the control group, was not sterilized; the second group was packaged and sterilized at <5° C. by gamma irradiation at a dose of 2.5 to 4.0 mRad; and the third group was packaged and sterilized at 25° C. by gamma irradiation at a dose of 2.5 to 4.0 mRad. Cooling during the <5° C. sterilization was accomplished by the use of Cold Packs coupled and specialized packaging [product available as WMX, from DHL, Paris, France]. Temperature was monitored by a 3M MonitorMark Temperature Indicator, St. Paul, Minn., ensuring the temperature did not exceed 5° C. Turning to FIG. 1 significant aggregation was observed by microscopy in both drug loaded and unloaded (no pharmaceutically active agent) microspheres which were sterilized by gamma irradiation at 25° C. By contrast, the both the drug loaded and unloaded microspheres which were sterilized at <5° C. have significantly less aggregation. FIGS. 2-4 detail the particle diameter distribution of the various batches before and after gamma irradiation. A significant increase in average particle diameter and in the breadth of the distribution particle diameters is observed for all batches of microspheres which were sterilized at 25° C. By contrast those batches of microspheres which were sterilized at a reduced temperature show an essentially identical volume and number average particle size distribution with their non-sterilized counterparts. These results demonstrate that the aggregation of PLGA microspheres due to gamma irradiation is essentially eliminated by reducing the temperature of the microspheres to around 5° C. or less during the sterilization.

While not intending to be limited or bound in any way by theory, it is generally accepted in the art that irradiation of polymers is generally harmful to the properties of said polymer. It is also generally accepted in the art that the degradation of polymers due to irradiation is generally due to the introduction of high energy species such as radicals, ions, and thermally and electronically excited species, which are also highly reactive. These highly reactive species induce aggregation of microparticles, as is the case in this experiment, but also contribute to a number of other degradation processes possible in polymers. This experiment demonstrates that reducing the temperature retards the degradation of polymeric material by retarding the formation of the highly reactive species, reducing the rate of the degradation processes, or both. Therefore, degradation of polymeric material due to irradiative sterilization will be retarded by reducing the temperature of the sterilization process for any polymer where sterilization causes such problematic degradation.

### EXAMPLE 2

A dose of tazarotene (1 mg) contained in the poly(lactide-co-glycolide) microsphere suspension of Example containing 1 is injected subconjunctivally into a patient suffering from retinitis pigmentosa. Maintenance of vision or a slowing of the progression of vision loss is observed for the duration of treatment.

1. A sterilized polymeric material for use in a body of a mammal wherein said polymeric material is sterilized by irradiation at a temperature below 25° C.

2. The sterilized polymeric material of claim 1 which further comprises a therapeutically active agent.

3. The sterilized polymeric material of claim 1 wherein said temperature is at or below about 5° C.

4. The sterilized polymeric material of claim 2 which comprises polymeric microspheres, microparticles, microcapsules, or implants.

5. The sterilized polymeric material of claim 2 which comprises polymeric microspheres, microparticles, or microcapsules.

6. The sterilized polymeric material of claim 5 which comprises polymeric microparticles.

7. The sterilized polymeric material of claim 5 which comprises polylactide-co-glycolide or polylactic acid.

8. The sterilized polymeric material of claim 5 which comprises polylactide-co-glycolide.

9. The sterilized polymeric material of claim 5 wherein said temperature is below about 15° C.

10. The sterilized polymeric material of claim 5 wherein said temperature is below about 10° C.

11. The sterilized polymeric material of claim 5 wherein said composition temperature is at or below about 5° C.

12. The sterilized polymeric material of claim 5 which is sterilized by gamma irradiation at a dose of about 1.5 to about 4.0 mRad.

13. The sterilized polymeric material of claim 5 wherein said therapeutically active agent comprises a retinoid, a prostaglandin, a tyrosine kinase inhibitor, a glucocorticoid, an androgenic steroid, an estrogenic steroid, a non-estrogenic steroid, an intracellular adhesion molecule inhibitor or an alpha-2-adrenergic agonist.

14. The sterilized polymeric material of claim 5 wherein said therapeutically active agent comprises a retinoid.

15. The sterilized polymeric material of claim 5 wherein said therapeutically active agent comprises tazarotene.

16. A method of sustained delivery of a therapeutically active agent to a mammal comprising administering a sterilized polymeric material comprising said therapeutically active agent to said mammal, wherein the polymeric material is sterilized by irradiation at a temperature below 25° C.

17. The method of claim 16 wherein the sterilization by irradiation comprises gamma irradiation.

18. The method of claim 17 wherein said temperature is below about 15° C.

19. The method of claim 17 wherein said temperature is below about 10° C.

20. The method of claim 17 wherein said temperature is below about 5° C.

21. The method of claim 17 wherein said temperature is from −25° C. to 5° C.

22. A method of sterilizing a polymeric material for use in a body of a mammal comprising irradiating said polymeric material at a temperature below 25° C.

23. The method of claim 22 wherein the polymeric material further comprises a therapeutically active agent.

24. The method of claim 22 wherein said temperature is below about 15° C.

25. The method of claim 22 wherein said temperature is below about 10° C.

26. The method of claim 22 wherein said temperature is below about 5° C.

27. A composition comprising sterilized polymeric microparticles and a therapeutically active agent for use in a body of a mammal wherein said polymeric material is sterilized by irradiation with external cooling of said polymeric material during sterilization.

28. The composition of claim 27 wherein said composition is suitable for sustained delivery of said therapeutically active agent.

29. The composition of claim 27 wherein the temperature of said polymeric material at the end of the sterilization process is about 10° C. to about 50° C. lower than said temperature would be in the absence of external cooling.

30. The composition of claim 27 wherein the temperature of said polymeric material at the end of the sterilization process is about 20° C. to about 50° C. lower than said temperature would be in the absence of external cooling.

31. The composition of claim 27 wherein the temperature of said polymeric material at the end of the sterilization process is about 50° C. or more lower than said temperature would be in the absence of external cooling.

32. A method of sterilizing a polymeric material for use in a body of a mammal comprising irradiating said polymeric material with external cooling of the polymeric material.

33. The method of claim 32 wherein the temperature of said polymeric material at the end of the sterilization process is about 10° C. to about 50° C. lower than said temperature would be in the absence of external cooling.

34. The method of claim 32 wherein the temperature of said polymeric material at the end of the sterilization process is about 20° C. to about 50° C. lower than said temperature would be in the absence of external cooling.

35. The method of claim 32 wherein the temperature of said polymeric material at the end of the sterilization process is about 50° C. or more lower than said temperature would be in the absence of external cooling.

36. The method of claim 16 wherein the polymeric material comprises polylactide-co-glycolide or polylactic acid.

37. The method of claim 16 wherein the polymeric material comprises polylactide-co-glycolide.

38. The composition of claim 27 wherein the polymeric material comprises polylactide-co-glycolide or polylactic acid.

39. The composition of claim 27 wherein the polymeric material comprises polylactide-co-glycolide.
